(19) United States
(12) Patent Application Publication
(10) Pub. No.: US 2022/0128536 A1
(43) Pub. Date: Apr. 28, 2022

(54) DETECTION METHOD FOR ORIGIN DETERMINATION BASED ON GAS CHROMATOGRAPHY-ION MOBILITY SPECTROMETRY (GC-IMS)

(71) Applicant: Jiaying University, Meizhou City (CN)

(72) Inventors: Liangzheng Xu, Meizhou City (CN); Hanqing Lv, Meizhou City (CN); Yimin An, Meizhou City (CN); Wei Kuang, Meizhou City (CN); Huina Liu, Meizhou City (CN)

(21) Appl. No.: 17/511,854

(22) Filed: Oct. 27, 2021

(30) Foreign Application Priority Data
Oct. 27, 2020 (CN) 202011167930.8

(51) Int. Cl. G01N 33/02; G01N 33/00; G01N 30/72; G01N 27/622

(52) U.S. Cl. CPC G01N 33/025; G01N 27/622; G01N 30/7206; G01N 33/0047

(57) ABSTRACT

A detection method for origin determination is provided. A gas chromatography-ion mobility spectrometry (GC-IMS) technology is adopted to obtain GC-IMS diagrams by detecting types and contents of volatile organic compounds in fruits. After analysises, fingerprint diagrams and principal component analysis (PCA) diagrams of the volatile organic compounds in the fruits are obtained. Origins of fruits can be determined based on the fingerprint diagrams and the PCA diagrams. By datumizing the traditional smelling method, it can assist scientific researchers and producers or businesses to identify origins or specific producing areas of fruits.

### TECHNICAL FIELD

The invention relates to detection methods for quickly determining origins of fruits and vegetables.

### DESCRIPTION OF RELATED ART

China has a vast territory and a wide variety of fruits, and geographical distributions of fruits are concentrated according to different types of fruits and climatic conditions required by themselves. In China, tropical and subtropical fruits such as coconuts, mangoes, pineapples, longans, lychees, pomelos and bananas are most afraid of 0° C. low temperature, so they are only distributed in southern China. Subtropical fruits such as citruses and loquats can withstand light cold, but they will still cause serious freezing damage at low temperatures around −9° C. or even below, so they are generally only distributed in the south of Qinling mountain-Huaihe River. A temperate zone in the north of the Qinling mountain-Huaihe River is rich in temperate fruits such as apples, pears, persimmons and grapes. For areas in the north of the Great Wall of China and northern Xinjiang province, it is difficult for temperate fruits such as apples to grow because of the severe cold in winter. In recent years, by using methods such as grafting and hybridization, apples have begun to expand further north.

At present, people usually judge an origin of a fruit based on the fruit's type and behavior. This traditional method of determining the origin is greatly affected by personal subjective factors and is closely related to personal experience. Therefore, how to use a standardized method to quickly identify origins of various fruits is an urgent problem to be solved.

### SUMMARY

In view of the fact that there is no standardized method in the existing technology to quickly detect origins of fruits, the invention aims at providing detection methods for quickly determining origins of fruits.

In order to achieve the above objective, the invention provides technical solutions as follows.

In particular, a detection method for determining origins of fruits, may include following steps:

step 1, collecting fruit samples from two or more different origins;

step 2, dividing each of the fruit samples into two parts of peel and pulp, cutting the peel and the pulp of each of the fruit samples into pieces, and numbering the peel and the pulp of each of the fruit samples;

step 3, weighing 1 g of the peel and 1 g of the pulp of each of the fruit samples, putting the 1 g of peel and the 1 g of pulp of each of the fruit samples into 20 mL of headspace bottles respectively, and tightening caps of the headspace bottles;

step 4, heating and vibrating each of the headspace bottles in an an incubator at 40° C. for 10 min, taking 500 μL of gas from a headspace of the headspace bottle and injecting into a gas chromatography-ion mobility spectrometry (GC-IMS) instrument;

step 5, controlling flow rates of carrier gas and drift gas as per a preset procedure in the GC-IMS instrument and thereby obtaining GC-IMS diagrams of volatile organic compounds of the peels and the pulps of the fruits samples after being placed for 20 min;

step 6, performing analysis and comparison on the GC-IMS diagrams to obtain fingerprint diagrams and principal component analysis (PCA) diagrams of the volatile organic compounds of the peels and the pulps of the fruits samples and thereby establish a fruit origin classification model; and

step 7, determining origins of fruits according to the fruit origin classification model.

In an embodiment, the analysis and comparison in the step 6 are performed on the GC-IMS diagrams by using softwares including Laboratory Analytical Viewer (LAV), Reporter plug-in, Gallery Plot plug-in, Dynamic PCA plug-in, and GC×IMS Library Search.

In an embodiment, specific steps for establishing the fruit origin classification model in the step 6 include:

S61, performing principal component analysis on the peels or the pulps of the fruit samples by using the Dynamic PCA plug-in to obtain a principal component GC-IMS diagram;

S62, performing statistical analysis on volumes of compounds in the obtained principal component GC-IMS diagram;

S63, setting peak volume data as an independent variable X and classification labels as Y, and selecting 70% of the fruit samples as a training dataset and 30% of the fruits samples as a test dataset;

S64, standardizing data in the training dataset, and then using a 10-fold cross-validation to build a model by selecting a certain number of latent variables according to a cross-validation error rate;

S65, calculating a cross-validation error of the built model to the samples in the training dataset and minimizing the cross-validation error of the built model, and finally establishing the fruit origin classification model; and

S66, taking data of the samples in the test dataset as inputs of the fruit origin classification model for prediction and calculating a classification accuracy rate, to evaluate a predictive ability of the fruit origin classification model.

In an embodiment, specific steps of determining origins of fruits according to the fruit origin classification model in the step 7 include:

S71, acquiring values of data of a to-be-tested fruit obtained by observation or measurement;

S72, inputting the values of the data of the to-be-tested fruit into the fruit origin classification model and performing calculation processing; and

S73, obtaining a classification result of the data of the to-be-tested fruit.

Compared with the prior art, the invention can achieve the following beneficial effects that:

First, by using the method of the invention, the instrument can quickly detect the volatile organic compounds in the peels and pulps of the fruits after the samples each are injected into the headspace bottle without needs of vacuumizing and sample pre-processing.

Second, by using the GC-IMS instrument, it can quickly detect the volatile organic compounds in peel and pulp of a fruit to be tested, that is, the odor of the fruit, and by datumizing the traditional smelling method, it can assist scientific researchers and producers or businesses in the identification of origin or specific producing area of a fruit.

### BRIEF DESCRIPTION OF DRAWINGS

FIG. 1 shows diagrams of volatile organic compounds of peels of pomelos from different origins for direct comparison of differences.

FIG. 2 shows diagrams of volatile organic compounds of peels of pomelos from different origins, in which one is used as a reference and the others each are deducted by the reference, for difference comparison.

FIG. 3A shows three-dimensional fingerprint diagrams of volatile organic compounds of peels of pomelo samples from Guangdong.

FIG. 3B shows three-dimensional fingerprint diagrams of volatile organic compounds of peels of pomelo samples from Guangxi.

FIG. 4A shows three-dimensional fingerprint diagrams of volatile organic compounds of pulps of pomelo samples from Guangdong.

FIG. 4B shows three-dimensional fingerprint diagrams of volatile organic compounds of pulps of pomelo samples from Guangxi.

FIG. 5 shows a PCA diagram of peels of pomelo samples from Guangdong and Guangxi.

FIG. 6 shows a PCA diagram of pulps of pomelo samples from Guangdong and Guangxi.

FIG. 7 shows a qualitative analysis diagram of volatile organic compounds of a peel of a pomelo sample from Guangxi.

FIG. 8 shows a qualitative analysis diagram of volatile organic compounds of a pulp of a pomelo sample from Guangxi.

### DETAILED DESCRIPTION OF EMBODIMENTS

The invention will be described below in detail with reference to specific embodiments. It should be noted that for those skilled in the art, several modifications and changes can be made without departing from the principle of the invention, which should also be regarded as belonging to the protection scope of the invention

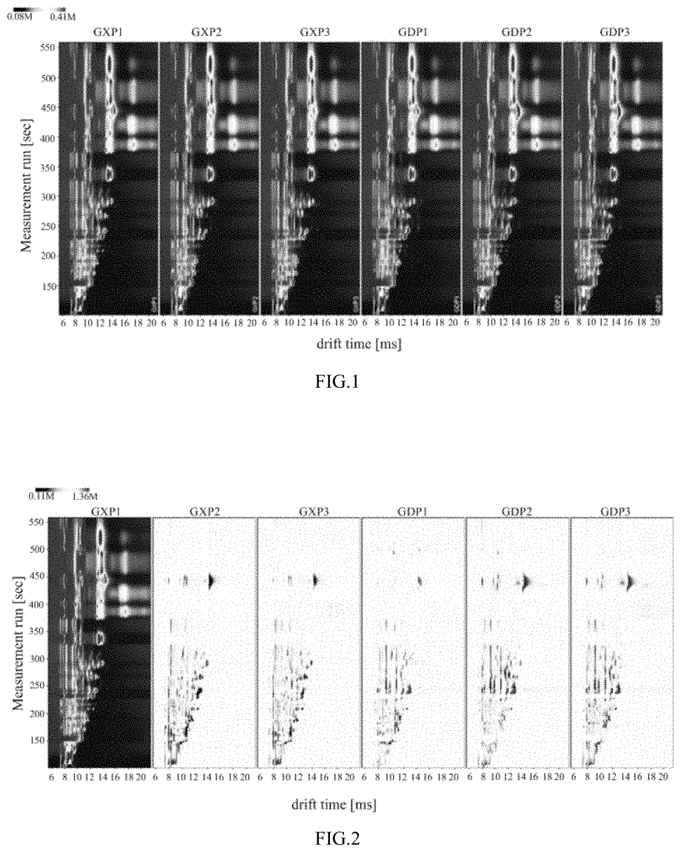

Referring to FIG. 1 through FIG. 5, it can be concluded that a detection method for quickly determining origins of fruits may include the following steps.

Step 1, collecting several fruit samples from two or more different origins. Preferably, three repeated samples are taken from each of the origins, that is, each of the origins is taken out three fruit samples for testing.

Step 2, dividing each of the fruit samples into two parts of peel and pulp, cutting the peel and the pulp of each of the fruit samples into small pieces, and numbering the peel and the pulp of each of the fruit samples. The repeated fruit samples are numbered as 1, 2 and 3 respectively.

Step 3, weighing 1 gram (g) of the peel and 1 g of the pulp of each of the fruit sample, putting each of them directly into a 20 milliliters (mL) headspace bottle, and tightening a cap of the headspace bottle.

Step 4, heating and vibrating the headspace bottle in an incubator at 40° C. for 10 min, and then automatically taking 500 microliters (μL) of gas from a headspace of the headspace bottle by a syringe and injecting into a gas chromatography-ion mobility spectrometry (GC-IMS) instrument. When heating for incubation, the higher the temperature, the more volatile organic compounds released from the sample. In order to be closer to the true state of the sample, try to make an incubation temperature close to room temperature when fruits and vegetables are heated.

Step 5, controlling flow rates of carrier gas and drift gas according to a preset procedure in the GC-IMS instrument, and thereby obtaining GC-IMS diagrams of volatile organic compounds of the peels and the pulps of the fruit samples after being placed for 20 minutes.

Step 6, performing analysis and comparison on the obtained GC-IMS diagrams of the fruit samples to obtain fingerprint diagrams and principal component analysis (PCA) diagrams of the volatile organic compounds of the peels and the pulps of the fruit samples and thereby establish a fruit origin classification model. When establishing the data model, the more sample data, the more reliable authenticity of an established Shatian pomelo model.

Step 7, determining origins of fruits according to the fruit origin classification model.

In an embodiment, analysis softwares used for the analysis and comparison in step 6 may include LAV, Reporter plug-in, Gallery Plot plug-in, Dynamic PCA plug-in, and GC×IMS Library Search.

The LAV (laboratory analytical viewer) software is used to view the analysis diagrams. Each point in the GC-IMS diagrams represents one type of volatile organic compound, which can be quantitatively analyzed after establishing a standard curve.

The Reporter plug-in is used to directly compare differences of GC-IMS diagrams between samples (two-dimensional top view and three-dimensional diagrams).

The Gallery Plot plug-in is used to perform comparison of fingerprint diagrams, intuitively and quantitatively compare differences of volatile organic compounds of different samples.

The Dynamic PCA plug-in is used to perform dynamic principal component analysis, and specifically used to perform cluster analysis on the samples and quickly determine types of unknown samples.

For the GA×IMS Library Search application software, built-in NIST database and IMS database thereof can perform qualitative analysis of substances, and users can use standard products to expand the databases according to their needs.

A basic idea of establishing the fruit origin classification model of the invention may be: adopting a partial least square discriminant analysis (PLS-DA) to establish the fruit origin classification model. The PLS-DA is a commonly used multivariate statistical analysis method for discriminant analysis, which is able to judge/determine how to classify researched objects based on the observed or measured values of several variables.

In an embodiment, specific steps for establishing the fruit origin classification model for fruits from different origins described in step 6 are as follows:

S61, performing PCA analysis on the peels or the pulps of the samples through the Dynamic PCA plug-in, to obtain a principal component GC-IMS diagram;

S62, performing statistical analysis on volumes of compounds in the obtained GC-IMS diagram;

S63, setting peak volume data as independent variable X and classification labels as Y, and selecting 70% of the samples as a training dataset and 30% of the samples as a test dataset;

S64, standardizing data in the training dataset, and then using a 10-fold cross-validation to build a model by selecting an appropriate number of latent variables based on a cross-validation error rate or an explained variance (≥85% or ≥90%);

S65, calculating a cross-validation error of the built model to the samples in the training dataset and minimizing the cross-validation error of the built model, and finally establishing the fruit origin classification model; and

S66, taking data of several sample in the test dataset as model inputs for prediction and calculating a classification accuracy rate, to evaluate a predictive ability of the model.

In an embodiment, specific operation steps of step 7 may include:

S71, acquiring values of data of a to-be-tested fruit obtained by observation or measurement;

S72, inputting the acquired values of the data of the fruit into the established fruit origin classification model and performing calculation processing; and

S73, obtaining a classification result of the data of the fruit.

### Example

In a use of the method of the invention, firstly, pomelos from two origins of Guangdong and Guangxi are selected as samples, and three samples are taken from each of the origins as repeated samples.

Subsequently, the pomelo samples each are cut into two parts of peel and pulp (flesh) with a knife, and numbered individually as that: GXP-Guangxi peel, GXR-Guangxi pulp, GDP-Guangdong peel, GDR-Guangdong pulp, the repeated samples are numbered as 1, 2, 3 respectively.

Afterwards, the peels of the pomelo samples are preferably used for analysis. In particular, for each pomelo sample, 1 g of the pomelo peel is weighed and placed directly into a 20 mL of headspace bottle, and then a cap of the headspace bottle is tightened. A procedure of instrument is set, the headspace bottle is heated and vibrated in an incubator at 40° C. for 10 minutes, and after that, a syringe is used to automatically take 500 μL gas from a headspace of the headspace bottle and automatically inject into a GC-IMS instrument for analysis.

Then, the GC-IMS instrument controls flow rates of carrier gas and drift gas according to the set procedure, and after 20 min, a gas chromatography-ion mobility spectrometry diagram of volatile organic compounds of the pomelo peel of each sample is obtained.

Finally, analysis softwares of the GC-IMS instrument are used to analyze the obtained GC-IMS diagrams of the peels of the pomelo samples.

Specifically, first, the Reporter plug-in is used to directly compare the volatile organic compounds in the peels of the pomelo samples of Guangdong and Guangxi, as shown in FIG. 1. It can be found that the ordinate represents the retention time of the gas chromatogram, and the abscissa represents the ion drift time. In the whole image, each the vertical line at the abscissa 8 is the RIP peak (reactive ion peak, not normalized), each of points on both sides of the RIP peak represents one kind of volatile organic compound, different colors represent different concentrations of substances.

In order to acquire differences among different samples more clearly by comparison, the GC-IMS diagram GXP1 of one sample (Guangxi peel No. 1) is selected as a reference, and the GC-IMS diagrams of the other samples after being deducted by the reference are shown in FIG. 2. It can be found that, if two volatile organic compounds are the same, the background after deduction is white, light black means a concentration of the substance is higher than that of the reference, deep black means a concentration of the substance is lower than that of the reference. As seen from the above direct comparison and difference comparison diagrams that the volatile organic compounds in peels of the pomelo samples from different origins are very different. For better comparison, peaks of these volatile organic compounds are selected to form fingerprint diagrams of the samples for comparison.

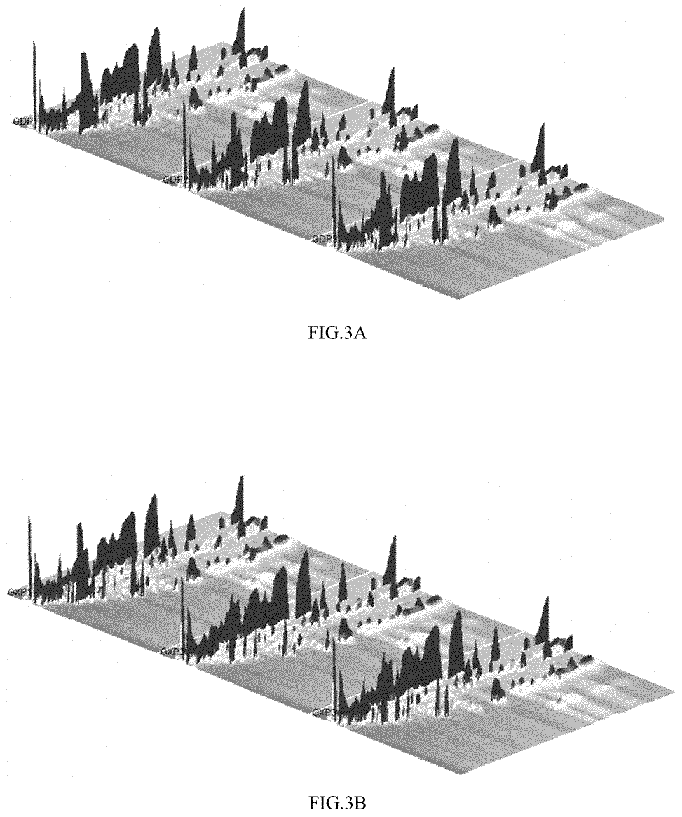

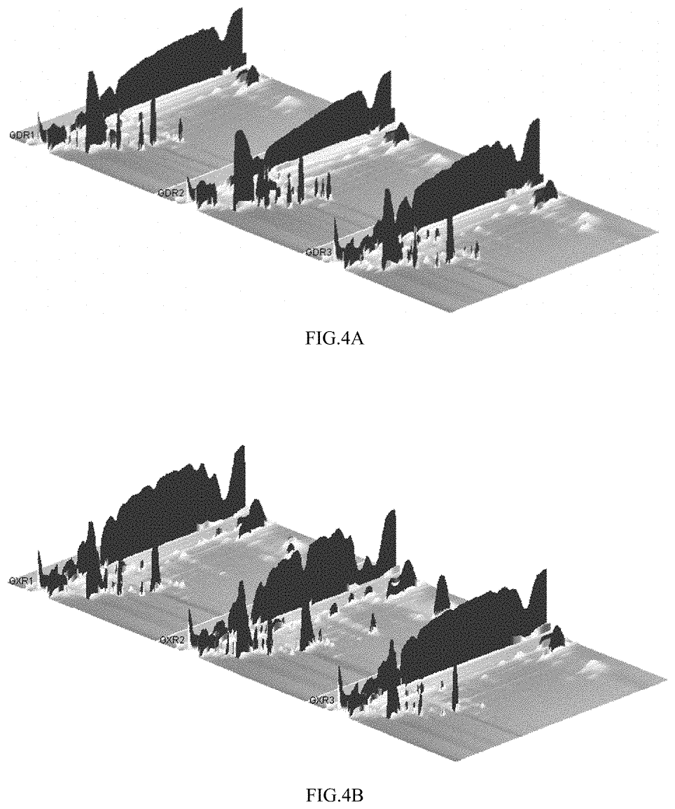

Second, the Gallery Plot plug-in is used to compare fingerprint diagrams of volatile organic compounds of the peels of the pomelo samples from Guangdong and Guangxi, and three-dimensional diagrams as generated are shown in FIG. 3A and FIG. 3B. Likewise, fingerprint diagrams of volatile organic compounds of the pulps of the pomelo samples from Guangdong and Guangxi are compared, and three-dimensional diagrams as generated are shown in FIG. 4A and FIG. 4B. It can be found that, there are differences in complete volatiles information of various samples and the volatile organic compounds among the samples, and peak height signals of volatile organic compounds in the respective origins also are different. Table 1 below shows the comparison of the peak heights of GXP (Guangxi peels) and GDP (Guangdong peels). It can be clearly seen that the volatile organic compounds of GXP (Guangxi peels) and GDP (Guangxi peels) are different.

TABLE 1

| | | “alpha- | “alpha- | “3- | | | |
|---|---|---|---|---|---|---|---|
| | “limonene” | pinene” | pinene” | octanone” | “limonene” | “16” | “107” |
| GXP2 | 147 | 387 | 184 | 91 | 184 | 168 | 26 |
| GXP3 | 140 | 401 | 203 | 115 | 177 | 166 | 35 |
| GXP1 | 152 | 402 | 203 | 104 | 184 | 171 | 25 |
| GDP1 | 154 | 411 | 186 | 106 | 184 | 163 | 30 |
| GDP2 | 165 | 384 | 180 | 98 | 184 | 148 | 38 |
| GDP3 | 166 | 437 | 210 | 113 | 185 | 169 | 35 |

Likewise, analysis of fingerprint diagrams of volatile organic compounds of GXR (Guangxi pulps) and GDR (Guangdong pulps) of the samples can be performed, and peak height comparison of GXR (Guangxi pulps) and GDR (Guangdong pulps) can be obtained as shown in Table 2.

TABLE 2

| | “ethyl acetate” | “limonene” | “ethyl butanoate” |
|---|---|---|---|
| GXR1 | 2517 | 233 | 1553 |
| GXR2 | 2500 | 327 | 729 |
| GXR3 | 2481 | 311 | 1744 |
| GDR1 | 2510 | 332 | 1855 |
| GDR2 | 2511 | 300 | 1849 |
| GDR3 | 2469 | 330 | 1700 |

Therefore, origins of this kind of fruit can be identified by using characteristic markers of origins.

Third, the Dynamic PCA plug-in is used to perform dynamic principal component analysis (PCA) on the peels (GXP and GDP) of the pomelo samples from different origins. As shown in FIG. 5, it can be seen that the three GDPs (Guangdong peels) are clustered together, this kind of fruit is in the main producing area of Guangdong and has a better aggregation effect on PCA. This kind of fruit in Guangxi is relatively scattered in producing area, and the aggregation effect on PCA is relatively scattered.

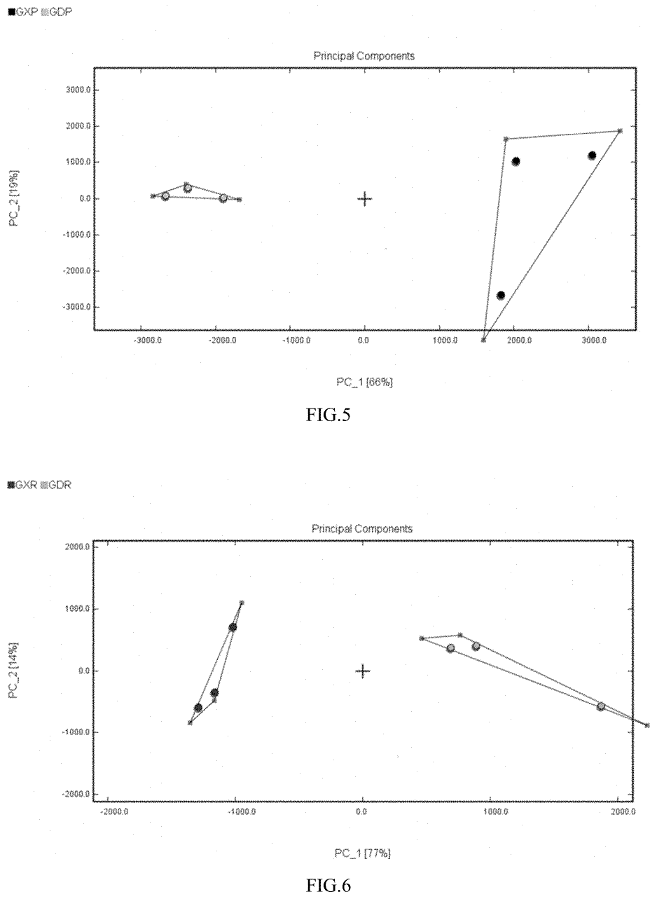

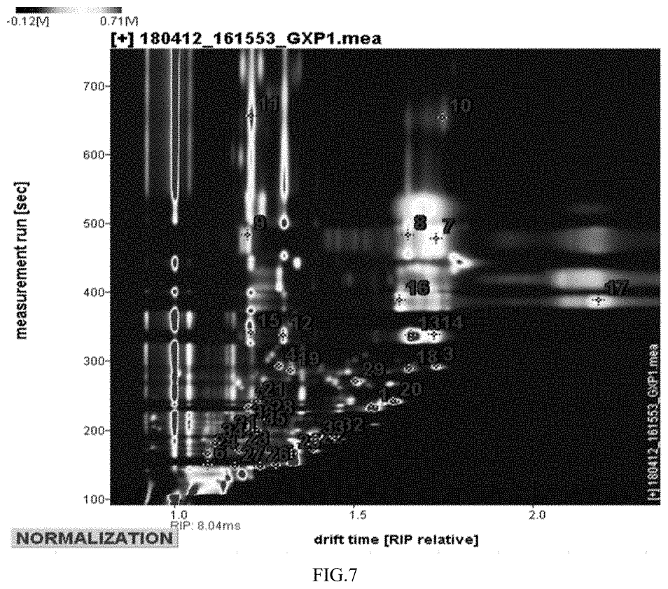

Similarly, PCA analysises are performed on the pulps (GXR and GDR) of the pomelo samples from different origins. As shown in FIG. 6, it can be seen that the three GXR (Guangxi pulps) are clustered together, this kind of fruit is in the main producing area of Guangxi and has a better aggregation effect on PCA. This kind of fruit in Guangdong is relatively scattered in producing area, and the aggregation effect on PCA is relatively scattered.

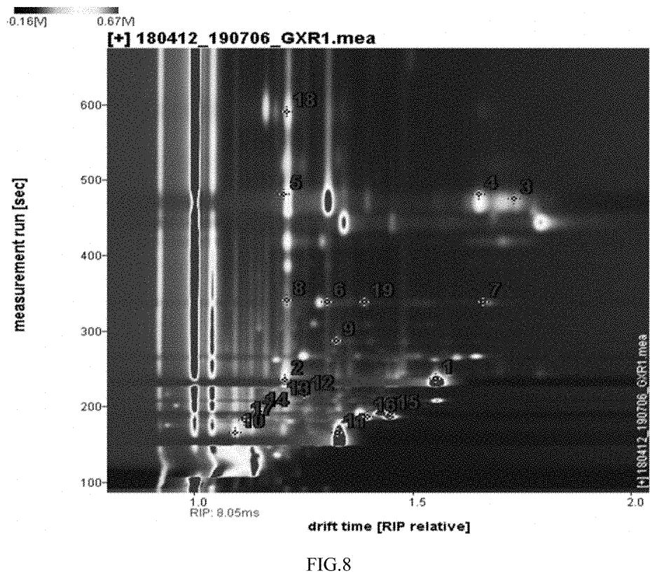

Fourth, the GC×IMS Library Search is used to perform qualitative analysis on volatile organic compounds in GXPs of the pomelo samples. As shown in FIG. 7, contents of the volatile organic compounds in the peels of the pomelo samples can be seen, and a compound list corresponding to FIG. 7 is shown in Table 3.

TABLE 3

| Compound | CAS# | Formula | MW | RI | Rt[sec] | Dt[RIPrel] |
|---|---|---|---|---|---|---|
| Ethyl butanoate | C105544 | C6H1202 | 116.2 | 793.2 | 233.084 | 15.548 |
| ethyl butanoate | C105544 | C6H1202 | 116.2 | 792.4 | 232.599 | 12.096 |
| 3-Methybutyl acetate | C123922 | C7H1402 | 130.2 | 878.2 | 292.666 | 17.353 |
| 3-Methybutyl acetate | C123922 | C7H1402 | 130.2 | 877.6 | 292.181 | 1.297 |
| Isobutanal | C78842 | C4H80 | 72.1 | 578.1 | 149.077 | 12.862 |
| Isobutanal | C78842 | C4H80 | 72.1 | 579.8 | 149.565 | 10.956 |
| Limonene | C138863 | C10H16 | 136.2 | 1020.2 | 477.293 | 17.355 |
| Limonene | C138863 | C10H1B | 136.2 | 1023.0 | 482.787 | 16.545 |
| Limonene | C138863 | C10H16 | 136.2 | 1023.0 | 482.787 | 1.208 |
| Linalool | C78706 | C10H180 | 154.3 | 1100.2 | 654.171 | 17.517 |
| Linalool | C78706 | C10H180 | 154.3 | 1101.1 | 656.283 | 12.155 |
| alpha-Pinene | C80568 | C10H16 | 136.2 | 925.7 | 337.837 | 13.069 |
| alpha-Pinene | C80568 | C10H16 | 136.2 | 925.7 | 337.837 | 1.664 |
| alpha-Pinene | C80568 | C10H16 | 136.2 | 927.0 | 339.237 | 17.295 |
| alpha-Pinene | C80568 | C10H16 | 136.2 | 928.4 | 340.637 | 12.152 |
| 3-octanone | C106683 | C8H160 | 128.2 | 968.2 | 388.231 | 16.312 |
| 3-octanone | C106683 | C8H160 | 128.2 | 968.2 | 388.231 | 21.882 |
| n-Hexanol | C111273 | C6H140 | 102.2 | 870.6 | 288.976 | 1.658 |
| n-Hexanol | C111273 | C6H140 | 102.2 | 867.1 | 286.337 | 13.281 |
| ethyl butyrate | C105544 | C6H1202 | 116.2 | 803.1 | 242.048 | 16.137 |
| ethyl butyrate | C105544 | C6H1202 | 116.2 | 803.1 | 242.048 | 12.316 |
| 1-butanol | C71363 | C4H100 | 74.1 | 655.9 | 171.628 | 13.928 |
| 1-butanol | C71363 | C4H100 | 74.1 | 654.2 | 171.099 | 11.838 |
| Butanal | C123728 | C4H80 | 72.1 | 635.5 | 165.262 | 10.962 |
| Ethyl Acetate | C141786 | C4H802 | 88.1 | 635.5 | 165.262 | 10.962 |
| Ethyl Acetate | C141786 | C4H802 | 88.1 | 637.1 | 165.75 | 13.296 |

The same method is used to perform qualitative analysis on the volatile organic compounds in GXRs of the pomelo samples. As shown in FIG. 8, contents of the volatile organic compounds in the pulps of the samples can be seen, and a compound list corresponding to FIG. 8 is shown in Table 4.

TABLE 4

| Compound | CAS# | Formula | MW | RI | Rt[sec] | Dt[RIPrel] |
|---|---|---|---|---|---|---|
| Ethyl butanoate | C105544 | C6H1202 | 116.2 | 793.2 | 233.084 | 15.548 |
| ethyl butanoate | C105544 | C6H1202 | 116.2 | 792.4 | 232.599 | 12.096 |
| Limonene | C138863 | C10H16 | 136.2 | 1020.2 | 477.293 | 17.355 |
| Limonene | C138863 | C10H16 | 136.2 | 1023.0 | 482.787 | 16.545 |
| Limonene | C138863 | C10H16 | 136.2 | 1023.0 | 482.787 | 1.208 |
| alpha-Pinene | C80568 | C10H16 | 136.2 | 925.7 | 337.837 | 13.069 |
| alpha-Pinene | C80568 | C10H16 | 136.2 | 925.7 | 337.837 | 1.664 |
| alpha-Pinene | C80568 | C10H16 | 136.2 | 928.4 | 340.637 | 12.152 |
| n-Hexanol | C111273 | C6H140 | 102.2 | 867.1 | 286.337 | 13.281 |
| Butanal | C123728 | C4H80 | 72.1 | 635.5 | 165.262 | 10.962 |
| Ethyl Acetate | C141786 | C4H802 | 88.1 | 635.5 | 165.262 | 10.962 |
| Ethyl Acetate | C141786 | C4H802 | 88.1 | 637.1 | 165.75 | 13.296 |
| 1-pentanol | C71410 | C5H120 | 88.1 | 758.1 | 215.719 | 12.547 |
| Hexan-2-one | C591786 | C5H120 | 100.2 | 745.6 | 209.028 | 12.013 |
| Ethyl propanoate | C105373 | C5H1002 | 102.1 | 712.0 | 192.855 | 11.512 |
| ethyl propanoate | C105373 | C5H1102 | 102.1 | 704.5 | 189.62 | 14.518 |
| ethyl acrylate | C140885 | C5H802 | 100.1 | 697.2 | 186.588 | 13.983 |
| 2-pentanone | C107879 | C5H100 | 86.1 | 683.3 | 181.177 | 11.182 |
| Linalool | C78706 | C10H180 | 154.3 | 1073.1 | 590.516 | 12.156 |
| Heptanol | C53535334 | C7H160 | 116.2 | 926.4 | 338.577 | 13.923 |

By using the above four analysis and comparison methods, the fingerprint diagrams and the principal component analysis diagram of the volatile organic compounds in peels of the pomelo samples from different origins can be obtained, and origins of fruits can be determined based on the fingerprint diagrams and the principal component analysis (PCA) diagram as obtained.

In summary, by using the method of the invention, the detection instrument can quickly detect the volatile organic compounds in the peels and pulps of the fruits after the samples each are injected into the headspace bottle without needs of vacuumizing and sample pre-processing, and moreover, it can analyze samples from different angles by using the LAV (Laboratory Analytical Viewer), the three plug-ins and the GC×IMS Library Search, and the following information can be obtained.

(1) GXPs (Guangxi peels) and GDPs (Guangdong peels) each contain many kinds of volatile organic compounds. The contents of some of the volatile organic compounds are higher in GXPs (Guangxi peels), and GXPs (Guangxi peels) contain characteristic volatile components. Based on this, it can be determined/judged whether the origin of this kind of fruit is Guangxi. The contents of certain volatile organic compounds in GDPs (Guangdong peels) are higher than that of GXPs (Guangxi peels). According to the comprehensive information of these volatile organic compounds, the origin or the specific producing area of this kind of fruit can be determined.

(2) Types of volatile organic compounds in the edible part (pulp) of this kind of fruit are significantly less than those in the peel. For example, the contents of pleasant aroma components such as limonene, limon alcohol, and pinene are very small in the pulp, but are very high in the peel. The origin or specific producing area of this kind of fruit can also be judged by the types of total volatile organic compounds in the pulp.

(3) No matter using PCA analysis on the peel or the pulp, fruits from different origins can be distinguished. After establishing a fruit analysis model for fruits from different origins, origins or specific producing areas of fruits can be quickly identified.

(4) By using the GC-IMS (Gas Chromatography-Ion Mobility Spectrometry) instrument, it can quickly detect the volatile organic compounds in peel and pulp of a fruit to be tested, that is, the odor of the fruit, and by datumizing the traditional smelling method, it can assist scientific researchers and producers or businesses in the identification of origin or specific producing area of a fruit.

What is claimed is:

1. A detection method for determining origins of fruits, comprising:
collecting fruit samples from two or more different origins;
dividing each of the fruit samples into two parts of peel and pulp, cutting the peel and the pulp of each of the fruit samples into pieces, and numbering the peel and the pulp of each of the fruit samples;
weighing a first amount of the peel and a second amount of the pulp of each of the fruit samples, putting the first amount of peel and the second amount of pulp of each of the fruit samples into headspace bottles respectively, and tightening caps of the headspace bottles;
heating and vibrating each of the headspace bottles in an incubator at a target temperature for a first predetermined time, taking a target volume of gas from a headspace of the headspace bottle and injecting into a gas chromatography-ion mobility spectrometry (GC-IMS) instrument;
controlling flow rates of carrier gas and drift gas as per a preset procedure in the GC-IMS) instrument and thereby obtaining GC-IMS diagrams of volatile organic compounds of the peels and the pulps of the fruits samples after being placed for a second predetermined time;
performing analysis and comparison on the GC-IMS diagrams to obtain fingerprint diagrams and principal component analysis (PCA) diagrams of the volatile organic compounds of the peels and the pulps of the fruits samples and thereby establish a fruit origin classification model; and
determining origins of fruits according to the fruit origin classification model.

2. The detection method as claimed in claim 1, wherein the analysis and comparison are performed on the GC-IMS diagrams by using softwares including Laboratory Analytical Viewer (LAV), Reporter plug-in, Gallery Plot plug-in, Dynamic PCA plug-in, and GC×IMS Library Search.

3. The detection method as claimed in claim 2, wherein specific steps for establishing the fruit origin classification model comprise:
performing principal component analysis on the peels or the pulps of the fruit samples by using the Dynamic PCA plug-in to obtain a principal component GC-IMS diagram;
performing statistical analysis on volumes of compounds in the obtained principal component GC-IMS diagram;
setting peak volume data as an independent variable X and classification labels as Y, and selecting 70% of the fruit samples as a training dataset and 30% of the fruits samples as a test dataset;
standardizing data in the training dataset, and then using a 10-fold cross-validation to build a model by selecting a certain number of latent variables according to a cross-validation error rate;
calculating a cross-validation error of the built model to the samples in the training dataset and minimizing the cross-validation error of the built model, and finally establishing the fruit origin classification model; and
taking data of the samples in the test dataset as inputs of the fruit origin classification model for prediction and calculating a classification accuracy rate, to evaluate a predictive ability of the fruit origin classification model.

4. The detection method as claimed in claim 1, wherein specific steps of determining origins of fruits according to the fruit origin classification model comprise:
acquiring values of data of a to-be-tested fruit obtained by observation or measurement;
inputting the values of the data of the to-be-tested fruit into the fruit origin classification model and performing calculation processing; and
obtaining a classification result of the data of the to-be-tested fruit.

5. The detection method as claimed in claim 1, wherein the first amount of the peel is 1 gram (g) of the peel, the second amount of the pulp is 1 g of the pulp, the headspace bottle is a 20 milliliters (mL) of headspace bottle, the target temperature is 40° C., the first predetermined time is 10 minutes (min), the target volume of gas is 500 microliters (μL) of gas, and the second predetermined time is 20 min.

6. A detection method for determining origins of fruits, comprising:
collecting fruit samples from two or more different origins;
dividing each of the fruit samples into two parts of peel and pulp, and numbering the peel of each of the fruit samples;
weighing a target amount of the peel of each of the fruit samples, putting the target amount of peel into a headspace bottle, and tightening a cap of the headspace bottle;
heating and vibrating the headspace bottle in an incubator at a target temperature for a first predetermined time, taking a target volume of gas from a headspace of the headspace bottle and injecting into a gas chromatography-ion mobility spectrometry (GC-IMS) instrument;
controlling flow rates of carrier gas and drift gas as per a preset procedure in the GC-IMS) instrument and thereby obtaining a GC-IMS diagram of volatile organic compounds of the peel of each of the fruits samples after being placed for a second predetermined time;
performing analysis and comparison on the GC-IMS diagrams of the fruit samples to obtain fingerprint diagrams and a principal component analysis (PCA) diagram of the volatile organic compounds of the peels of the fruits samples and thereby establish a fruit origin classification model; and
determining origins of fruits according to the fruit origin classification model.

7. The detection method as claimed in claim 6, wherein the target amount of the peel is 1 g of the peel, the headspace bottle is a 20 mL of headspace bottle, the target temperature is 40° C., the first predetermined time is 10 min, the target volume of gas is 500 μL of gas, and the second predetermined time is 20 min.

8. A detection method for determining origins of fruits, comprising:
collecting fruit samples from two or more different origins;
dividing each of the fruit samples into two parts of peel and pulp, and numbering the pulp of each of the fruit samples;
weighing a target amount of the pulp of each of the fruit samples, putting the target amount of pulp into a headspace bottle, and tightening a cap of the headspace bottle;
heating and vibrating the headspace bottle in an incubator at a target temperature for a first predetermined time, taking a target volume of gas from a headspace of the headspace bottle and injecting into a gas chromatography-ion mobility spectrometry (GC-IMS) instrument;
controlling flow rates of carrier gas and drift gas as per a preset procedure in the GC-IMS) instrument and thereby obtaining a GC-IMS diagram of volatile organic compounds of the pulp of each of the fruits samples after being placed for a second predetermined time;
performing analysis and comparison on the GC-IMS diagrams of the fruit samples to obtain fingerprint diagrams and a principal component analysis (PCA) diagram of the volatile organic compounds of the pulps of the fruits samples and thereby establish a fruit origin classification model; and
determining origins of fruits according to the fruit origin classification model.

9. The detection method as claimed in claim 8, wherein the target amount of the pulp is 1 g of the pulp, the headspace bottle is a 20 mL of headspace bottle, the target temperature is 40° C., the first predetermined time is 10 min, the target volume of gas is 500 μL of gas, and the second predetermined time is 20 min.
